(19) United States
(12) Patent Application Publication
(10) Pub. No.: US 2013/0231547 A1
(43) Pub. Date: Sep. 5, 2013

(54) NOVEL PLANAR SMALL ELECTRODE SENSOR FOR SKIN IMPEDANCE MEASUREMENT AND SYSTEM USING THE SAME

(71) Applicants: Min Soo Kim, Daegu-si (KR); Suk Tae Seo, Ulsan-si (KR); Chang Sik Son, Daegu-si (KR); Hee Joon Park, Daegu-si (KR); Yoon Nyun Kim, Daegu-si (KR)

(72) Inventors: Min Soo Kim, Daegu-si (KR); Suk Tae Seo, Ulsan-si (KR); Chang Sik Son, Daegu-si (KR); Hee Joon Park, Daegu-si (KR); Yoon Nyun Kim, Daegu-si (KR)

(73) Assignee: KEIMYUNG UNIVERSITY INDUSTRY ACADEMIC COOPERATION FOUNDATION, Daegu-si (KR)

(21) Appl. No.: 13/882,497

(22) PCT Filed: Sep. 15, 2011
(86) PCT No.: PCT/KR2011/006811

§ 371 Date: Apr. 29, 2013

(30) Foreign Application Priority Data
Nov. 23, 2010 (KR) 10-2010-0116765

(51) Int. Cl. A61B 5/053

(52) U.S. Cl. CPC A61B 5/0532; A61B 5/0536
USPC 600/393

(57) ABSTRACT

Disclosed are a planar small electrode sensor for skin impedance measurement and a system using the same. The sensor include: a semiconductor substrate; an insulating layer formed on the substrate; and at least one pair of electrodes which are symmetrically formed on the insulating layer with respect to a vertical central line of the insulating layer, where the at least one pair of electrodes includes a reference electrode and a measuring electrode.

### TECHNICAL FIELD

The present invention relates to a planar small electrode sensor for skin impedance measurement and a skin impedance measurement system using the same, and more particularly, to a novel planar small electrode sensor which is capable of detecting acupuncture points through skin impedance measurement and a skin impedance measurement system using the same.

### BACKGROUND ART

The dominant theorem of acupuncture mechanism is to acquire an effect by changing a particular brain's functioning through a physical (mechanical and/or electrical) stimulus of acupuncture points located over a human body. Connective tissues of particular acupuncture points and their neurological positions play a direct role in effectiveness of acupuncture, which relied on technique and experience of medical workers in the past.

In recent years, electrical impedance tomography (EIT) has been in the spotlight because of low hardware costs for system implementation and nondestructive measurement for objects to be measured. Although EIT provides spatial resolution of a restored image lower than those of X-ray and MRI tomography, it is being used as ancillary equipments in medical engineering since it provides high temporal resolution and guarantees safety of a human body.

Acupuncture points are anatomically distributed in a two-dimensional space and different human bodies have different distributions of nervous tissues and soft tissues. Therefore, finding positions of acupuncture points has tended to rely on experience of medical workers. EIT is to reinvent engineering analysis of acupuncture points.

EIT measures resistance of a body tissue after flowing current of several miliamperes and 10 to 100 kHz into the body tissue, from which it has been discovered that acupuncture points have impedance lower than those of other neighboring tissues. Based on such discovery, EIT detects electrical characteristics of body sections by flowing current into body parts through respective electrodes attached to the body parts in a sequential manner, measuring resistance of the body parts and forming an image pertinent to the measured resistance.

Conventional electrical impedance tomography (EIT) will be described below with reference to the accompanying drawings.

FIGS. 1 to 4 are views for explaining the principle of imaging resistance of the interior of a body in conventional EIT. As described above, EIT is a technology which detects electrical characteristics of body sections by flowing current into body parts through respective electrodes attached to the body parts in a sequential manner, measuring resistance of the body parts and imaging the measured resistance.

To this end, as shown in FIG. 1, input electrodes S and s and receiving electrodes R and r in the form of 2×2 matrix are attached to body tissues and then resistance of the body tissues is measured by flowing current into the body tissues through these electrodes. In FIG. 2, the input electrodes S and s are divided into horizontal input electrodes S1 and S2 and vertical input electrodes s1 and s2 and the receiving electrodes R and r are divided into horizontal receiving electrodes R1 and R2 and vertical receiving electrodes r1 and r2.

Subsequently, as shown in FIG. 2, horizontal impedance is measured by flowing current from the horizontal input electrodes S1 and S2 to the horizontal receiving electrodes R1 and R2. Then, as shown in FIG. 3, vertical impedance is measured by flowing current from the vertical input electrodes s1 and s2 to the vertical receiving electrodes r1 and r2.

These measurements allow estimation of a distribution of impedance values of body parts by inverse-nonlinear data processing. This EIT apparatus has a shape of annular cylinder and measures resistance by flowing current into body parts, such as a trunk, a wrist, an ankle and so on, attached with the apparatus. For example, the horizontally and vertically-measured resistance corresponds to the total sum of resistance of body tissues and may be used to detect a distribution of resistance of tissues projecting onto a section. As an alternative, after a distribution of resistance is known, a distribution of voltage may be calculated based on an intensity of current and a position of an equipotential line may be presented based on the calculated distribution of voltage.

### DISCLOSURE

### Technical Problem

However, such a conventional EIT apparatus has been known to have the following problems. First, since this apparatus has a distribution of cylindrical electrodes enclosing the human body, skeletal tissues of the human body are unnecessarily imaged, which spends much time to process data. Secondly, since this apparatus has the distribution of cylindrical electrodes enclosing the human body, there may occur a thickness difference between body parts, for example between a wrist and a waist, arranged with the same number of electrodes, which may result in deterioration of resolution of an acupuncture point at the thicker body part with a wider distance between the electrodes.

As such, since acupuncture points, in other words, biological active points (BAPS), have characteristics of high local temperature, electrical potential and electric capacitance and low resistance as compared to biological inactive points, measurement of skin impedance is required to make detailed analysis on electrical characteristics of skin.

However, the above conventional skin impedance measurement system and method has problems in various respects such as pressure applied to electrodes, dependency of skin impedance on current and voltage, characteristics of skin conductance at acupuncture points, etc. In addition, this system and method has a further problem that it requires unpractical time and stabilization period (period required to diffuse a sufficient electrolytic gel into skin) taken to measure the skin impedance using electrodes from the viewpoint of medical treatment.

### Technical Solution

To overcome the above problems, it is an object of the invention to provide a system which is capable of easily finding biological active points (BAPs) or acupuncture points as well as providing high reliability and precision for research and investigation of such biological active points (acupuncture points).

It is another object of the invention to provide a method of fabricating a simple and inexpensive sensor and system which is capable of providing measurement data with high resolution.

To achieve the above objects, according to a first aspect of the present invention, there is provided a small electrode sensor for skin impedance measurement, including: a semiconductor substrate; an insulating layer formed on the substrate; and at least one pair of electrodes which are symmetrically formed on the insulating layer with respect to a vertical central line of the insulating layer, where the at least one pair of electrodes includes a reference electrode and a measuring electrode.

Preferably, the small electrode sensor further includes an adhesion layer formed between the insulating layer and the at least one pair of electrodes, and four pairs of electrodes are arranged on a circumference having a predetermined radius in a central portion.

According to a second aspect of the present invention, there is provided a system for measuring skin impedance, including: the above-described small electrode sensor; a circuit part including resistors and a capacitor connected in series or in parallel; a power source which applies a voltage or current; an amplifier which amplifies a signal; and a control and display unit which controls the system and displays a measured signal.

Preferably, the system is an apparatus for finding positions of acupuncture points.

According to a third aspect of the present invention, there is provided a method of fabricating a skin impedance measuring sensor having at least one pair of electrodes, including the steps of: (a) forming an insulating layer on a substrate; (b) applying a photoresist on the central portion of the insulating layer; (c) forming an adhesion layer on the insulating layer and the photoresist; (d) forming a metal electrode layer on the adhesion layer; and (e) forming electrodes by removing the photoresist and an upper layer using a lift-off method.

Preferably, the step (e) of forming electrodes includes forming at least one pair of electrodes including a measuring electrode and a reference electrode, which are disposed with an interval with respect to a central point, the step (c) includes forming a chromium adhesion layer by sputtering, and the step (d) includes forming a metal electrode layer by thermal vapor deposition.

### Advantageous Effects

According to the present invention, by measuring absolute values and phases of skin impedances at different frequencies, it is possible to find acupuncture points easily as well as provide high reliability and precision in research and investigation of skin biological active points (BAPs) (acupuncture points) of skin.

In addition, it is possible to provide a method of easily fabricating a simple and inexpensive sensor and system which is capable of providing measurement data with high resolution.

### DESCRIPTION OF DRAWINGS

FIGS. 1 to 4 are views for explaining the principle of imaging resistance of the interior of a body in conventional electrical impedance tomography (EIT).

FIG. 5 is a sectional view of a skin impedance measuring sensor according to an embodiment of the invention.

FIG. 6 is a photograph of a skin impedance measuring sensor according to an embodiment of the invention.

FIG. 7 is a block diagram of a skin impedance measuring system according to an embodiment of the invention.

FIGS. 8a to 8e are views for explaining a fabricating process of a skin impedance measuring sensor according to an embodiment of the invention.

FIG. 9 is a schematic view for explaining a three-electrode measuring method to measure skin impedance.

FIGS. 10a and 10b are graphs showing a frequency vs. impedance locus of BAPs and non-BAPs measured in a left arm of a subject using the system according to the embodiment of the invention.

FIGS. 11a and 11b are graphs showing a frequency vs. impedance locus of BAPs and non-BAPs measured in a right arm of the subject using the system according to the embodiment of the invention.

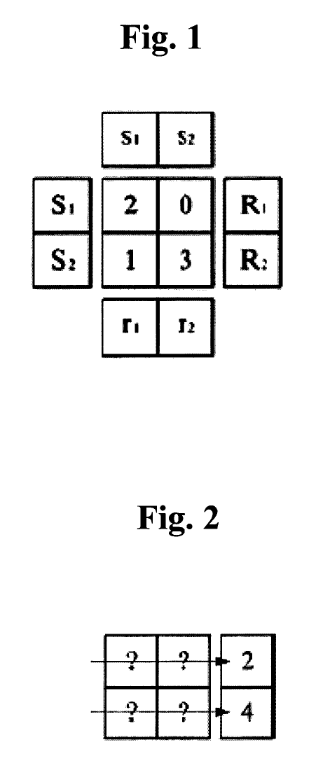

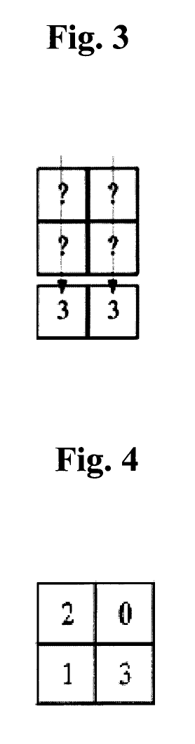

### BEST MODE

Hereinafter, preferred embodiments of the present invention will be described in detail with reference to the accompanying drawings.

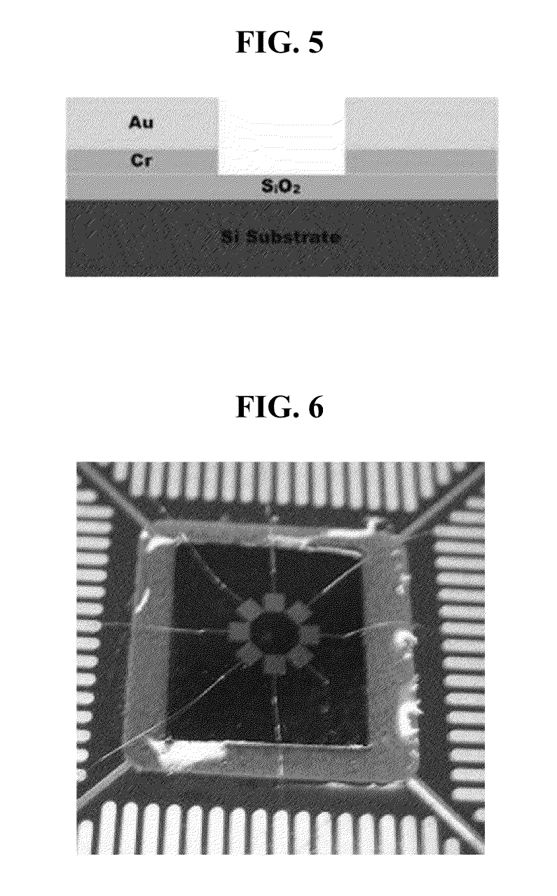

FIG. 5 is a sectional view of a skin impedance measuring sensor according to an embodiment of the invention, and FIG. 6 is a photograph of a skin impedance measuring sensor according to an embodiment of the invention. As shown in FIGS. 5 and 6, the sensor has a structure including a silicon substrate, an insulating layer, an adhesion layer, and a pair of electrode layer which is made of gold and formed with an interval with respect to the vertical central line of the silicon substrate.

As shown in FIG. 6, an embodiment of the invention involves a novel small planar electrode sensor (NPSES) having four pairs of electrodes, which is designed to measure electrical characteristics of biological active points (BAPS).

This small electrode semiconductor sensor is suggested to measure skin impedance to find an acupuncture point existing in a particular spot on skin, based on the fact that the acupuncture point has low skin impedance. That is, with the electrodes shown in FIG. 6 contacting a skin, current is applied to a reference electrode and a measuring electrode and impedance between the reference electrode and the measuring electrode is measured. The electrodes of the sensor as shown in FIG. 6 are arranged on the circumference having a certain radius with respect to the center point and tracks of the sensor has a small electrode structure where electrodes are arranged on a circle having a diameter of 2 mm.

In addition, as shown in FIG. 6, the skin impedance measuring sensor according to this embodiment is surrounded by four pairs of electrodes on the circumference of the central portion. This configuration facilitates measurement of skin impedance in all directions including the vertical and horizontal directions by sequentially applying current or voltage to each pair of electrodes. Then, the skin impedance is averaged to further improve the precision and reliability of measurement.

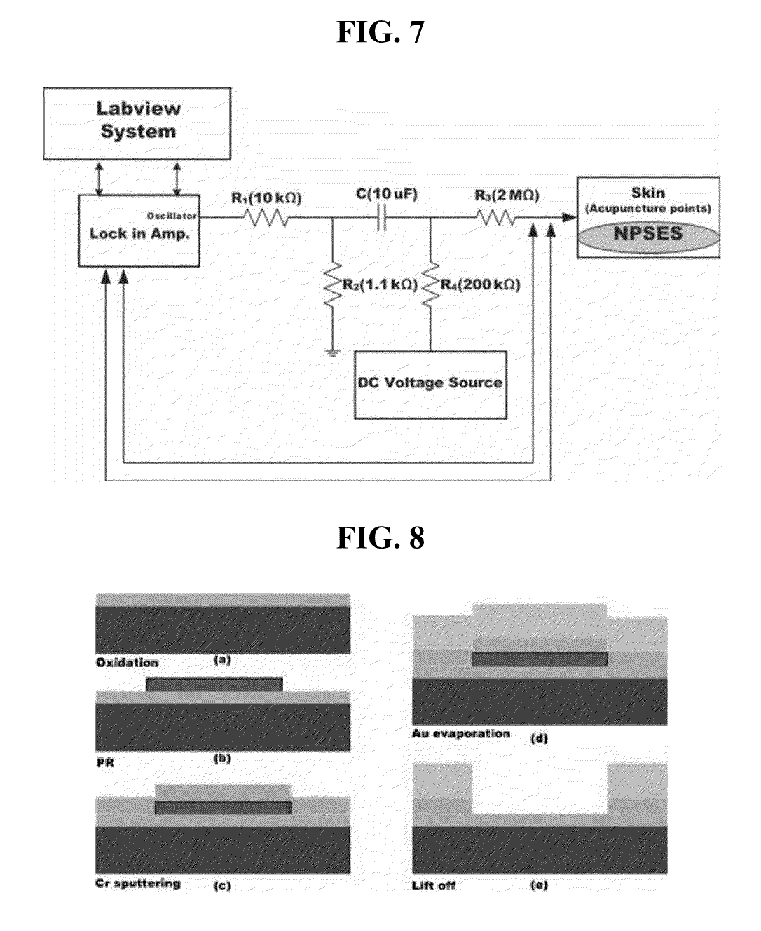

FIG. 7 is a block diagram of a skin impedance measuring system according to an embodiment of the invention. As shown in FIG. 7, the system includes the above-described skin impedance measuring sensor; a circuit part including resistors and a capacitor connected in series or in parallel; a power source which applies a voltage or current; an amplifier which amplifies a signal; and a control and display unit which controls the system and displays a measured signal.

In operation, minute current is sequentially applied from the power source (DC voltage source) to pairs of electrodes including the above-described measuring electrodes and reference electrodes in the above skin impedance measuring sensor. This minute current is generated by the circuit part including the resistors and capacitor connected in series or in parallel, and the signal measured by the sensor is amplified by the amplifier and then displayed on the control and display unit (for example, a Labview system). In this embodiment, the circuit part preferably includes four resistors and a 10 μF capacitor for application of the minute current and measurement of the signal. However, it is to be understood that the circuit part may be constructed in different ways if necessary.

FIGS. 8a to 8e are views for explaining a fabricating process of a skin impedance measuring sensor according to an embodiment of the invention. As shown in FIG. 8, this embodiment involves a method of fabricating a semiconductor device having at least one pair of electrodes, including the steps of: (a) forming an insulating layer on a substrate; (b) applying a photoresist on the central portion of the insulating layer; (c) forming an adhesion layer on the insulating layer and the photoresist; (d) forming a metal electrode layer on the adhesion layer; and (e) forming electrodes by removing the photoresist and an upper layer using a lift-off method.

In this embodiment, the insulating layer is formed by oxidizing a silicon oxide (SiO₂) substrate, where a silicon oxide layer is formed on the silicon substrate since this layer can remove leakage current leaked out of the substrate. The adhesion layer is formed of a chromium (Cr) layer. The adhesion layer is an intermediate layer formed to increase an adhesion between the SiO₂ layer and the metal electrode layer.

More specifically, a silicon oxide layer having a thickness of 1 μm is deposited on a substrate such as a Si wafer at about 1000° C. (FIG. 8a). Electrodes are patterned and formed with a gap therebetween by photolithography and lift-off process.

It is preferable that an adhesion layer such as a 70 nm-thick chromium layer is deposited by sputtering and a 250 nm-thick gold electrode layer is deposited by thermal vapor deposition. Gold (Au) is preferred material for the electrode layer because it has low resistance as an oxidized layer is not well formed by the surroundings.

Specifically, the insulating layer is formed on the substrate (FIG. 8a), photoresist (PR) is applied on the central portion of the insulating layer (FIG. 8b). The Cr adhesion layer is formed on the insulating layer and the PR layer by sputtering (FIG. 8c), and the Au electrode layer is formed on the adhesion layer by thermal vapor deposition (FIG. 8d). Thereafter, ultraviolet exposure is performed to remove the top of the PR layer (Lift-Off) (FIG. 8e), thereby completing an NPSES type skin impedance measuring sensor with a plurality of pairs of electrodes formed on the circumference, as shown in FIG. 6.

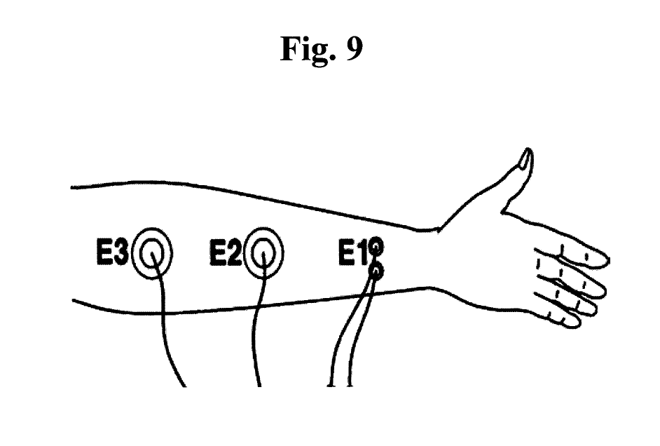

An exemplary experiment of finding acupuncture points for 5 healthy persons (at the age of 27 to 41) using the skin impedance measuring system fabricated through the fabricating method of the present invention will be now described. The experiment was made under the conditions of the room temperature of 21° C. and the humidity of 40%. The measuring method used was a 3-electrode method and FIG. 9 is a schematic view for explaining a three-electrode measuring method to measure skin impedance.

As shown in FIG. 9, the 3-electrode method has an advantage in measuring impedance of BAPs at skin. In this method, a measuring electrode (electrode E1 in FIG. 9) is used to apply a current and measure a voltage. This electrode contacts skin at a spot to be measured. An electrode E2 in FIG. 9 is a reference electrode which is adjacent to the measuring electrode E1. Finally, a ground electrode E3 in FIG. 9 is located apart by at least 10 cm from the measuring electrode E1.

That is, the skin impedance measuring system of this present has the structure where the plurality of pairs of electrodes including the measuring electrode and the reference electrode are arranged on the central portion of the measuring sensor (NPSES) in the circumference and the ground electrode is grounded in contact with one spot apart from the sensor, thereby allowing measurement of skin impedance using the 3-electrode method shown in FIG. 9. This 3-electrode method is beneficial in that BAPs at skin can be easily measured.

Experiment preparation and a measuring method to detect variation of impedance at acupuncture points using the skin impedance measuring system of the present invention and results of the measurement will be described.

In the present invention, skin impedance is measured by applying a sinusoidal wave current from a constant current source to skin. Here, a magnitude of applied current is 7.5 μA and its frequency is 0.1 Hz to 1.5 kHz.

The system of this invention is used to test reliability of micro electrodes and measure a relationship between skin resistance and reactance at BAPs of left and right arms.

Here, the skin resistance often means a friction generated by motion of ions such as sodium and chlorine ions. The reactance means polarization of normal molecules like cell membrane and protein in an electric field applied to dielectric filled between capacitor electrodes.

Since measurements of BAPs are different in a voltage between the measuring electrode and the reference electrode, a distance between the measuring electrode and the reference electrode is fixed and the ground electrode has to be relatively located in an inactive region. In order to measure BAPs at specified spots on skin, it is preferable that electrodes are connected when the ground electrode is fixed, and a signal continues to be applied during the measurement.

In this ways, in the present invention, absolute values and phase of impedance at all measuring frequencies are measured, real number parts and imaginary number parts of the impedance are calculated, and impedance vectors for current are calculated and depicted as a graph.

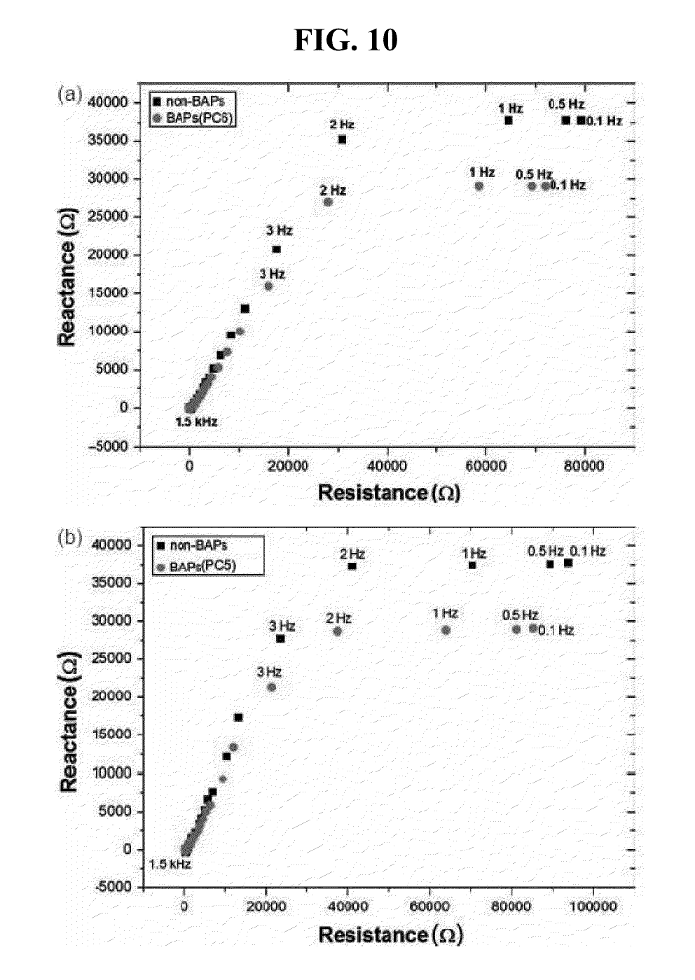

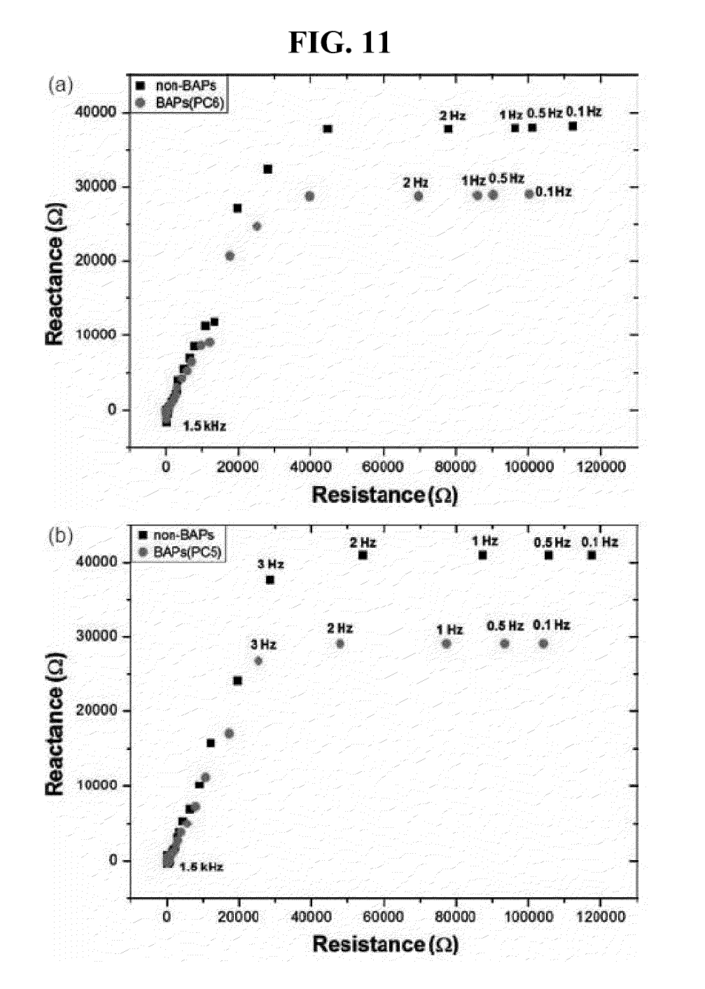

FIGS. 10 and 11 are graphs showing a frequency vs. impedance locus of BAPs and non-BAPs measured in left and right arms of subjects, respectively, using the system of this. FIGS. 10 and 11 show impedance measured ay BAPs PC5 and PC6 and non-BAPs for left and right arms of five subjects. PC5 (Jian Shi) and PC6 (Nei Guan) represent acupuncture points known in oriental medicine.

FIG. 10 shows that loci depend on a frequency and all frequency points form an arc. Measurements ay BAPs and non-BAPs are noticeable in a range of low frequencies but are insignificant in a range of high frequencies.

FIG. 11 shows results of measurement of skin impedance at BAPs and non-BAPs in the right arms of the subjects. FIG. 11 also shows results of measurement in a range of low frequencies of 0.1 to 10 Hz. This is because there is a noticeable difference between a skin impedance locus at BAPs and a skin impedance locus at non-BAPs in the range of low frequencies, as shown in FIGS. 10 and 11.

Accordingly, it is preferable to set the lower limit of the range of low frequencies applied to this invention to be above 0.1 Hz. The upper limit of the range of high frequencies is set to be 1.5 kHz or so. This is because there appears little difference in the range of high frequencies. In addition, since this invention uses small electrodes of size of several mm, it can be seen that real number parts and imaginary number parts of the impedance have large values.

In addition, as a result of application of the system of this invention, it can be seen that the range of frequencies preferred to perform reliable measurement for the skin impedance locus is 0.1 Hz to 1.5 kHz.

Although a few exemplary embodiments have been shown and described, it will be appreciated by those skilled in the art that adaptations and changes may be made in these exemplary embodiments without departing from the spirit and scope of the invention, the scope of which is defined in the appended claims and their equivalents.

### INDUSTRIAL APPLICABILITY

In this manner, in this invention, BAPs and non-BAPs can be distinguished from each other through spots showing a noticeable difference between skin impedances by measuring the absolute values and phases of the skin impedances of BAPs and non-BAPs at different frequencies. Accordingly, it is possible to find acupuncture points easily as well as provide high reliability and precision in research and investigation of BAPs (acupuncture points) using the system of this invention.

1-9. (canceled)

10. A small electrode sensor for skin impedance measurement, comprising:
a semiconductor substrate;
an insulating layer formed on the substrate; and
at least one pair of electrodes which are symmetrically formed on the insulating layer with respect to a vertical central line of the insulating layer,
where the at least one pair of electrodes includes a reference electrode and a measuring electrode.

11. The small electrode sensor according to claim 10, further comprising an adhesion layer formed between the insulating layer and the at least one pair of electrodes.

12. The small electrode sensor according to claim 10, wherein four pairs of electrodes are arranged on a circumference having a predetermined radius in a central portion.

13. A system for measuring skin impedance, comprising:
a small electrode sensor according to claim 10;
a circuit part including resistors and a capacitor connected in series or in parallel;
a power source which applies a voltage or current;
an amplifier which amplifies a signal; and
a control and display unit which controls the system and displays a measured signal.

14. A system for measuring skin impedance, comprising:
a small electrode sensor according to claim 11;
a circuit part including resistors and a capacitor connected in series or in parallel;
a power source which applies a voltage or current;
an amplifier which amplifies a signal; and
a control and display unit which controls the system and displays a measured signal.

15. A system for measuring skin impedance, comprising:
a small electrode sensor according to claim 12;
a circuit part including resistors and a capacitor connected in series or in parallel;
a power source which applies a voltage or current;
an amplifier which amplifies a signal; and
a control and display unit which controls the system and displays a measured signal.

16. The system according to claim 13, wherein the system is an apparatus for finding positions of acupuncture points.

17. The system according to claim 14, wherein the system is an apparatus for finding positions of acupuncture points.

18. The system according to claim 15, wherein the system is an apparatus for finding positions of acupuncture points.

19. A method of fabricating a skin impedance measuring sensor having at least one pair of electrodes, comprising the steps of:
(a) forming an insulating layer on a substrate;
(b) applying a photoresist on the central portion of the insulating layer;
(c) forming an adhesion layer on the insulating layer and the photoresist;
(d) forming a metal electrode layer on the adhesion layer; and
(e) forming electrodes by removing the photoresist and an upper layer using a lift-off method.

20. The method according to claim 19, wherein the step (e) of forming electrodes includes forming at least one pair of electrodes including a measuring electrode and a reference electrode which are disposed with an interval with respect to a central point.

21. The method according to claim 19, wherein the step (c) includes forming a chromium adhesion layer by sputtering.

22. The method according to claim 20, wherein the step (c) includes forming a chromium adhesion layer by sputtering.

23. The method according to claim 19, wherein the step (d) includes forming a metal electrode layer by thermal vapor deposition.

24. The method according to claim 20, wherein the step (d) includes forming a metal electrode layer by thermal vapor deposition.
